(19) United States
(12) Patent Application Publication
(10) Pub. No.: US 2025/0360123 A1
(43) Pub. Date: Nov. 27, 2025

(54) METHODS AND COMPOSITIONS FOR TREATING INHERITED RETINAL DEGENERATION

(71) Applicants: ONL Therapeutics, Inc., Ann Arbor, MI (US); The Regents of the University of Michigan, Ann Arbor, MI (US)

(72) Inventors: Andrew J. KOCAB, Farmington Hills, MI (US); David ZACKS, Ann Arbor, MI (US); Jingyu YAO, Ball Ground, GA (US)

(21) Appl. No.: 19/064,486

(22) Filed: Feb. 26, 2025

Related U.S. Application Data
(60) Provisional application No. 63/559,127, filed on Feb. 28, 2024.

(51) Int. Cl. A61K 31/4706; A61K 9/00; A61K 38/10; A61P 27/02

(52) U.S. Cl. CPC A61K 31/4706; A61K 9/0048; A61K 9/0053; A61K 38/10; A61P 27/02

(57) ABSTRACT

Provided herein are methods of treating inherited retinal degeneration in an eye of an individual, wherein the method comprises: administering: (i) hydroxychloroquine to the individual; and (ii) a Fas-inhibiting peptide to the eye, wherein the peptide comprises an amino acid sequence HHIYLGAVNYIY or variant sequence thereof, or a pharmaceutically acceptable salt thereof.

### CROSS-REFERENCE

This application claims the benefit of U.S. Provisional Patent Application No. 63/559,127 filed Feb. 28, 2024, which is herein incorporated by reference in its entirety for all purposes.

### BACKGROUND

Vision is generally dependent on maintaining the anatomical and histological integrity of the structures within the eye. Changes in anatomical and histological homeostasis can provide the basis for a decrease and/or loss in vision. Ocular diseases such as inherited retinal degeneration generally result in abnormal changes in the structure of the eye (e.g., retina) that can contribute to a decrease and/or loss in vision. Lesions in the retina generally arise due to injury or disease.

### SUMMARY

In some embodiments, provided and exemplified herein are methods of treating inherited retinal degeneration in an eye of an individual, wherein the method comprises: administering: (i) hydroxychloroquine to the individual; and (ii) a Fas-inhibiting peptide to the eye, wherein the peptide comprises an amino acid sequence HHIYLGAVNYIY or variant sequence thereof, or a pharmaceutically acceptable salt thereof. In certain embodiments, inherited retinal degeneration comprises retinitis pigmentosa. In certain embodiments, the individual has a P23H opsin mutation.

In some embodiments, provided and exemplified herein are methods of treating retinal cell death in an eye of an individual, wherein the method comprises: administering: (i) autophagy inhibitor to the individual; and (ii) a Fas-inhibiting peptide to the eye, wherein the peptide comprises an amino acid sequence HHIYLGAVNYIY or variant sequence thereof, or a pharmaceutically acceptable salt thereof. In certain embodiments, the retinal cell death comprises photoreceptor cell death, retinal epithelium cell death, or both photoreceptor cell death and retinal epithelium cell death.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides an experimental timeline for an investigation of the effect of delivery of an autophagy inhibitor (e.g., hydroxychloroquine, HCQ) in a mouse model of retinitis pigmentosa with genetically inactivated Fas receptors (e.g., FAS-Lpr/P23H mice);

FIG. 2A provides example images of the retina of FAS-Lpr/P23H mice labeled for a marker of cell death, terminal deoxynucleotidyl transferase (TdT) dUTP Nick-End Labeling (TUNEL), in an untreated (top) mouse and a mouse treated with an autophagy inhibitor (e.g., HCQ, bottom);

FIG. 2B provides a quantitation of TUNEL-positive cell counts in the retinas of FAS-Lpr/P23H mice that were either treated with an autophagy inhibitor (e.g., HCQ, left) or were untreated (right);

FIGS. 3A-3C provide a quantitation of cells expressing markers of cell inflammation, the inflammatory cytokine CCL-2 and CCL-3, in the retinas of FAS-Lpr/P23H mice that were either treated with an autophagy inhibitor (e.g., HCQ, FIG. 3A), FAS-Lpr/P23H mice were untreated (FIG. 3B), or in wildtype mice that were untreated (FIG. 3C);

FIGS. 4A-D provide the optic nerve layer (ONL) thickness, a measure of cell death, in 3-month old FAS-Lpr/P23H mice measured by optical coherence tomography (OCT) either 250 μm or 500 μm from the optic nerve in the inferior (inf; FIG. 4A and FIG. 4B) and superior (sup; FIG. 4C and FIG. 4D) retina;

FIGS. 5A-D provide the optic nerve layer (ONL) thickness, a measure of cell death, in 4-month old FAS-Lpr/P23H mice measured by optical coherence tomography (OCT) either 250 μm or 500 μm from the optic nerve in the inferior (inf; FIG. 5A and FIG. 5B) and superior (sup; FIG. 5C and FIG. 5D) retina;

FIG. 6 provides images of the retina of 3-month and 4-month old FAS-Lpr/P23H mice showing that delivery of an autophagy inhibitor preserves photoreceptor survival;

FIG. 7A provides example images showing increased expression of photoreceptor functional proteins in 3-month old FAS-Lpr/P23H mice treated with an autophagy inhibitor compared to untreated mice;

FIG. 7B provides example images showing increased expression of photoreceptor functional proteins in 4-month old FAS-Lpr/P23H mice treated with an autophagy inhibitor compared to untreated mice;

FIG. 8 shows the amplitude of scotopic and photopic electroretinogram (ERG) a- and b-waves, measurements of retinal health and function, showing that treatment with an autophagy inhibitor increases rod cell scotopic response in 3-month old FAS-Lpr/P23H mice;

FIG. 9 shows the amplitude of scotopic and photopic electroretinogram (ERG) a- and b-waves, measurements of retinal health and function, showing that treatment with an autophagy inhibitor increases rod cell scotopic response in 4-month old FAS-Lpr/P23H mice;

FIG. 10 provides an experimental timeline for an investigation of the effect of delivery of an autophagy inhibitor (e.g., HCQ) and a Fas inhibitor (e.g., ONL1204) in a mouse model of retinitis pigmentosa (e.g., P23H mice);

FIGS. 11A provides the optic nerve layer (ONL) thickness, a measure of cell death, in 3-month old P23H mice measured by optical coherence tomography (OCT) in mice treated with an autophagy inhibitor alone (circular markers) and with an autophagy inhibitor and a Fas inhibitor (square markers);

FIGS. 11B provides the optic nerve layer (ONL) thickness, a measure of cell death, in 3-month old P23H mice measured by optical coherence tomography (OCT) in mice treated with an autophagy inhibitor alone (circular markers) and with an autophagy inhibitor and a Fas inhibitor (square markers), showing that combined treatment better preserves ONL thickness than does autophagy inhibitor treatment alone;

FIG. 12 provides the amplitude of scotopic and photopic electroretinogram (ERG) a- and b-waves, measurements of retinal health and function, showing that treatment with combined autophagy inhibitor and Fas-inhibitor increases scotopic and photopic response in 3-month old P23H mice; and

FIG. 13 provides the amplitude of scotopic and photopic electroretinogram (ERG) a- and b-waves, measurements of retinal health and function, showing that treatment with combined autophagy inhibitor and Fas-inhibitor increases scotopic and photopic response in 4-month old P23H mice.

### DETAILED DESCRIPTION

Provided herein are Fas inhibitors useful for modulating (e.g., inhibiting, preventing, and/or reducing, etc.) Fas-mediated signaling. In certain instances, the Fas inhibitors useful in treating, inhibiting, preventing, and/or reducing Fas-mediated inflammation. In certain instances, inhibiting, preventing, and/or reducing Fas-mediated inflammation allows for the treatment and/or prevention of lesion growth within the retina (e.g., photoreceptors and/or retinal pigment epithelium).

In some embodiments, the Fas inhibitors described herein encompass Met-derived peptides and/or fragments thereof. In some embodiments, the Met protein, also called c-Met or hepatocyte growth factor receptor (HGF receptor), is encoded by the Met gene (NCBI Gene ID 4233, Location: NC_000007.14 (116672196 . . . 116798386), UniProtKB-P0858). The Met protein is comprised of two major subunits: the a and B subunits, and Met and fragments of Met, including the extracellular domain of Met and its a subunit, have been shown to bind to Fas and prevent cells from undergoing apoptosis. In some embodiments, the Fas inhibitor comprises a Fas-inhibiting peptide (e.g., Met-derived peptide and/or fragment thereof). In some embodiments, the Fas inhibitors described herein comprises a Met-derived compound comprising the amino acid acids HHIYLGAVNYIY (His-His-Ile-Tyr-Leu-Gly-Ala-Val-Asn-Tyr-Ile-Tyr) (e.g., SEQ ID NOs: 1-8). In some embodiments, the peptide comprises the amino acid sequence HHIYLGAVNYIY or a variant sequence thereof.

As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the Fas inhibitor peptides (e.g., a peptide comprising the amino acid sequence HHIYLGAVNYIY) comprises one or more naturally occurring amino acids. In some embodiments, the Fas inhibitor peptides (e.g., a peptide comprising the amino acid sequence HHIYLGAVNYIY) consists of naturally occurring amino acids. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the Fas inhibitor comprises a variant sequence of the peptide (e.g., a peptide comprising the amino acid sequence HHIYLGAVNYIY). In some embodiments, amino acid substitutions can be made in the sequence of any of the polypeptides described herein, without necessarily decreasing or ablating its activity. Accordingly, in some embodiments, the variant sequence comprises one or more amino acid substitutions. In some embodiments, the variant sequence comprises one amino acid substitution. In some embodiments, the variant sequence comprises two amino acid substitutions. In some embodiments, the variant sequence comprises three amino acid substitutions. In some embodiments, substitutions include conservative substitutions (e.g., substitutions with amino acids of comparable chemical characteristics). In some embodiments, a non-polar amino acid can be substituted and replaced with another non-polar amino acid, wherein non-polar amino acids include alanine, leucine, isoleucine, valine, glycine, proline, phenylalanine, tryptophan and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In some embodiments, the Fas inhibitor peptides (e.g., a peptide comprising the amino acid sequence HHIYLGAVNYIY) comprises one or more non-natural amino acids. In some embodiments, the Fas inhibitor peptides (e.g., a peptide comprising the amino acid sequence HHIYLGAVNYIY) consists of non-natural amino acids. As used herein, non-natural amino acids and/or unnatural amino acids include and/or refer to amino acid structures that cannot be generated biosynthetically in any organism using unmodified or modified genes from any organism. In some embodiments, non-natural amino acids and/or unnatural amino acids further include and/or refer to an amino acid residue that are not present in the naturally occurring (wild-type) Met protein sequence. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring (wild-type) Met protein sequence. A non-natural amino acid residue also can be incorporated such that a desired functionality is imparted to the apelin molecule, for example, the ability to link a functional moiety (e.g., PEG).

In some embodiments, a variant sequence comprises one or more amino acid deletions. In some embodiments, the variant sequence comprises one amino acid deletion. In some embodiments, the variant sequence comprises two amino acid deletions. In some embodiments, the variant sequence comprises three amino acid deletions. In some embodiments, the variant sequence comprises four amino acid deletions. In some embodiments, the variant sequence comprises one or more additional amino acids. In some embodiments, the additional amino acids are additional amino acids from the Met sequence. In some embodiments, the variant sequence comprises a substitution and a deletion. In some embodiments, the variant sequence comprises a substitution and one or more additional amino acids. In some embodiments, the substitution comprises a natural amino acid or a non-natural amino acid. In some embodiments, the variant sequence is a retro inverso amino acid sequence. In some embodiments, a variant sequence comprises one or more additional amino acid residues (e.g., one, two, or three additions) to the N or C terminus. In some embodiments, a variant sequence comprises one or more deletions (e.g., one, two, or three deletions) to amino acid residues at the N or C terminus.

In some embodiments, a variant sequence comprises one or two conservative amino acid substitutions. In some embodiments, the variant sequence further comprises a deletion of one or two terminal amino acids In some embodiments. In some embodiments, a variant sequence comprises one conservative amino acid substitution. In some embodiments, the variant sequence further comprises a deletion of one terminal amino acid. In some embodiments, a variant sequence comprises two conservative amino acid substitutions. In some embodiments, the variant sequence further comprises a deletion of two terminal amino acids. In certain embodiments, the variant sequence inhibits, reduces, or prevents cell death of cells treated with FasL.

Functionality of variant sequences of the peptide (e.g., a variant sequence of the amino acid sequence HHIYLGAVNYIY) can be determined by an in vitro assay. For example, in some embodiments, the variant sequence competes for binding to a Fas receptor (FasR) with Fas ligand (FasL). In some embodiments, the variant sequence inhibits, reduces, or prevents caspase 8 activation in cells treated with FasL (e.g., as measured by commercially available luminescent tetrapeptide cleavage assay kit (Promega, Madison, WI)). In some embodiments, the variant sequence inhibits, reduces, or prevents cell death of cells treated with FasL. By way of further example, in some embodiments, the variant sequence competes for binding to a Fas receptor (FasR) with a Fas-activating antibody (e.g., Fas-agonistic Jo2 monoclonal antibody (BD Biosciences, San Jose, CA)). In some embodiments, the variant sequence inhibits, reduces, or prevents caspase 8 activation in cells treated with a Fas-activating antibody (e.g., as measured by commercially available luminescent tetrapeptide cleavage assay kit (Promega, Madison, WI)). In some embodiments, the variant sequence inhibits, reduces, or prevents cell death of cells treated with a Fas-activating antibody. Accordingly, in some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with Fas ligand (FasL). In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents caspase 8 activation in cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents cell death of cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents caspase 8 activation in cells treated with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents cell death of cells treated with a Fas-activating antibody.

The peptide or a variant sequence thereof can further comprise one or more modifications. In some embodiments, the peptide (e.g., a comprising the amino acid sequence HHIYLGAVNYIY or a variant sequence thereof) comprises a modification. In some embodiments, the peptide is a modified peptide. As used herein, a modification or a modified peptide includes and/or refers to a modification of one or more amino acids in the peptide. In some embodiments, modifications species of stereoisomers. All stereoisomers of the above compounds are contemplated, either in admixture or in pure or substantially pure form. The compounds can have asymmetric centers at any of the atoms. Consequently, the peptide compounds or components thereof can exist in enantiomeric or diastereomeric forms or in mixtures thereof. The present invention contemplates the use of any racemates (i.e., mixtures containing equal amounts of each enantiomer), enantiomerically enriched mixtures (i.e., mixtures enriched for one enantiomer), pure enantiomers or diastereomers, or any mixtures thereof. The chiral centers can be designated as R or S or R, S or d, D, 1, L or d, 1, D, or L. Compounds comprising amino acid residues include residues of D-amino acids, L-amino acids, or racemic derivatives of amino acids. Compounds comprising sugar residues include residues of D-sugars, L-sugars, or racemic derivatives of sugars. Non-limiting examples of modifications are phosphorylation, glycosylation, ubiquitination, nitrosylation, methylation, acetylation, amidation, or lipidation. Modification can be introduced at the C-terminus of the peptide, the N-terminus of the peptide, or at any place in-between. Thus, a modification or a modified peptide includes and/or refers to modifications of the free amino-and/or carboxyl-terminal (N-terminus and C-terminus, respectively). In some embodiments, N-terminal modifications include but are not limited to acetylation, formylation, pyroglutamylation, carbamide addition, lipidation, sulfonamidation, and alkylamination. In some embodiments, C-terminal modifications include but are not limited to amidation, esterification, and incorporation of an aldehyde group. In some embodiments, the modification comprises amidation. In some embodiments, the amidation is at the c-terminus. In some embodiments, the modification comprises a retro inverso peptide (e.g., YIYNVAGLYIHH). In some embodiments, the modification altering the chirality of one or more amino acid residues of the peptide (e.g., L amino acid to D amino acid).

Accordingly, in an embodiment, provided herein are peptides comprising the sequence (a)-HHIYLGAVNYIY-(b) (SEQ ID NO: 1) or (a)-YIYNVAGLYIHH-(b) (SEQ ID NO: 2), or a variant sequence thereof, wherein:

(a) is —H, —OH, —NH₂, G¹(CH₂)ₙ—, R¹CONH—, or R²O—;

(b) is —H, —CH₂OH, —CH₂OR₂, —CHO, —CO₂R₂, —CONH₂, —CONHR₂, —CON(R³)₂, —CONH(CH₂)_{y}NR(³)₂, —(CH₂)ₙ—G¹, —COCH₂G¹, —CONHCH₂—G¹, —(CH₂)ₙNH₂, —(CH₂)ₙNHR₂, —(CH₂)ₙN(R³)₂, NH—Glu—His—OH, NH—Glu—His—NH₂, —Ala—His—NH₂, —Gly—His—NH₂, —NH—Glu—His—OH, —NH—Glu—His—NH₂, —Ala—His—NH₂, —Gly—His—NH₂, —NH—[D]Glu—[D]—His—OH, —NH—[D]Glu—[D]—His—NH₂, —[D]Ala—[D]—His—NH₂, —Gly[D]—His—NH₂, or —CONH(CH₂)ₙ—G²; G¹, at each occurrence, is independently —H, —C(═O)NH₂, —C(═O)NHR₂, —C(═O)N(R³)₂, C(═O)OR₂, or —C(═O)R¹; G² at each occurrence is a heterocyclic ring of 4-7 members comprising at least one tertiary amine functionality NR₂ within the ring, or a carbocyclic ring of 3-7 members substituted with —N(R³)₂;

R¹, at each occurrence, is independently H, C₁₋₆alkyl, —(CH₂)ₓ(OCH₂CH₂)ₘOR⁵, C₁₋₆-alkoxy or L;

R², at each occurrence, is independently C₁₋₆alkyl, C₂₋₆alkyl substituted with OR⁵ or NR⁵₂, —(CH₂)ₓ(OCH₂CH₂)ₘOR⁵ or L;

L, at each occurrence, is a multivalent polyethylene glycol derivative with 2-4 termini, each of which may be independently capped with H, R⁵;

R³, at each occurrence, is independently C₁₋₆alkyl, C₂₋₆alkyl substituted with OR⁵ or N(R⁵)₂, —(CH₂)ₓ(OCH₂CH₂)ₘOR⁵;

or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;

R⁴, at each occurrence, is independently C₁₋₆alkyl, C₁₋₆acyl, or —OPO₃(R⁵)₂;

R⁵, at each occurrence, is independently H or C₁₋₆alkyl;

R⁶, at each occurrence, is H, C₁₋₆alkyl, C₂₋₆hydroxyalkyl, C₁₋₆alkoxy-, C₁₋₆alkyl, or C₁₋₆acyl;

m=1-100;

n=0-3;

x=0-6; and

y=2-4,

wherein at most one of R¹ and R² is L.

In certain instances, provided herein are peptides comprising the structure of Formula I or Formula II, or a pharmaceutically acceptable salt thereof.

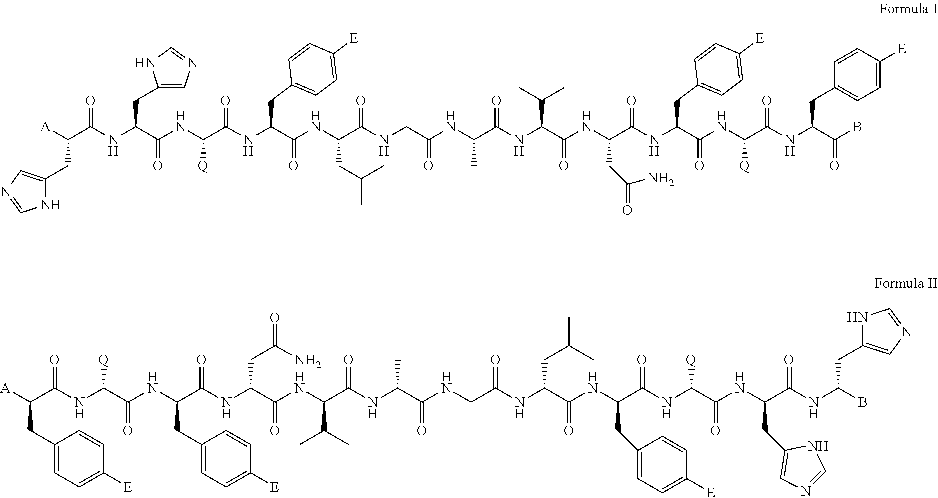

wherein:

A is H—, —OH, —NH₂, G¹(CH₂)ₙ—, R¹CONH—, or R²O—;

B is —H, CH₂OH, CH₂OR₂, —CHO, —CO₂R², —CONH₂, —CONHR², —CON(R³)₂, —CONH(CH₂)_{y}N(R³)₂, —(CH₂)ₙ—G¹, —COCH₂—G¹, —CONHCH₂—G¹, —(CH₂)ₙNH₂, —(CH₂), NHR², —(CH₂)ₙN(R³)₂NH—Glu—His—OH, NH—Glu—His—NH₂, —Ala—His—NH₂, —Gly—His—NH₂, NH—Glu—His—OH, NH—Glu—His—NH₂, —Ala—His—NH₂, —Gly—His—NH₂, NH—[D]Glu—[D]—His—OH, NH—[D]Glu—[D]—His—NH₂, —[D]Ala—[D]—His—NH₂, —Gly[D]—His—NH₂, or CONH(CH₂)ₙ—G²; E, at each occurrence, is independently —H, —OH, —OR⁴, SH, —SR⁴, or halogen; G¹, at each occurrence, is independently —H, —C(═O)NH₂, —C(═O)NHR², —C(═O)N(R³)₂, C(═O)OR², or —C(═O)R¹;

G² at each occurrence is a heteroalicyclic ring of 4-7 members comprising at least one tertiary amine functionality NR² within the ring, or a carbocyclic ring of 3-7 members substituted with N(R³)₂;

Q, at each occurrence, is independently, 1-propyl, 2-propyl, 2-methyl-prop-2-yl, C₃₋₆-cycloalkyl, C₄₋₆-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran-4-yltetrahydrothiopyran-2-yl, tetrahydrothiopyran-3-yl, tetrahydrothiopyran-4-yl or 1-CH(OR⁵)CH₃;

R¹, at each occurrence, is independently H, C₁₋₆alkyl, —(CH₂)ₓ(OCH₂CH₂)ₘOR⁵, C₁₋₆alkoxy or L;

R², at each occurrence, is independently C₁₋₆alkyl, C₂₋₆alkyl substituted with OR⁵ or N(R⁵)₂, —(CH₂)ₓ(OCH₂CH₂)ₘOR⁵or L;

L, at each occurrence, is a multivalent polyethylene glycol derivative with 2-4 termini, each of which may be independently capped with H, R⁵ or another molecule of the peptide of Formula I or II;

R³, at each occurrence, is independently C₁₋₆-alkyl, C₂₋₆-alkyl substituted with OR⁵ or N(R⁵)₂, —(CH₂)ₓ(OCH₂CH₂)ₘOR⁵;

or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;

R⁴, at each occurrence, is independently C₁₋₆alkyl, C₁₋₆acyl, or —OPO₃(R⁵)₂;

R⁵, at each occurrence, is independently H or C₁₋₆alkyl;

R⁶, at each occurrence, is H, C₁₋₆alkyl, C₂₋₆hydroxyalkyl, C₁₋₆alkoxy-, C₁₋₆alkyl, or C₁₋₆acyl;

m=1-100;

n=0-3;

x=0-6; and

y=2-4,

wherein at most one of R¹ and R² is L.

In some embodiments, provided is a peptide having the structure of Formula III or a pharmaceutically acceptable salt thereof:

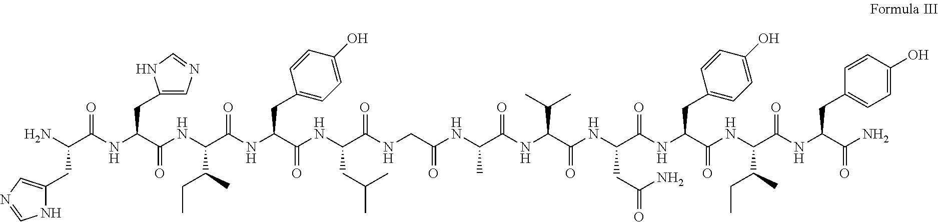 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

In some embodiments, provided is a peptide having the structure of Formula IV or a pharmaceutically acceptable salt thereof:

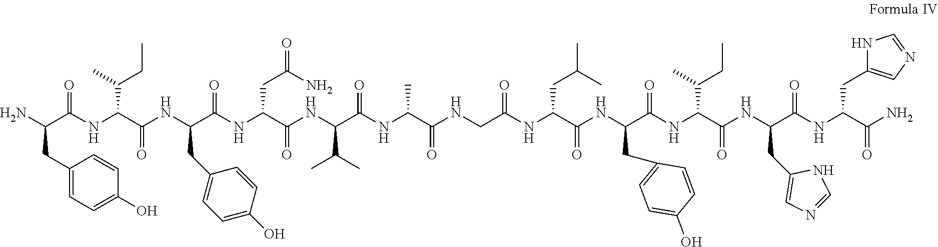 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

In some embodiments, provided is a peptide having the structure of Formula V or a pharmaceutically acceptable salt thereof:

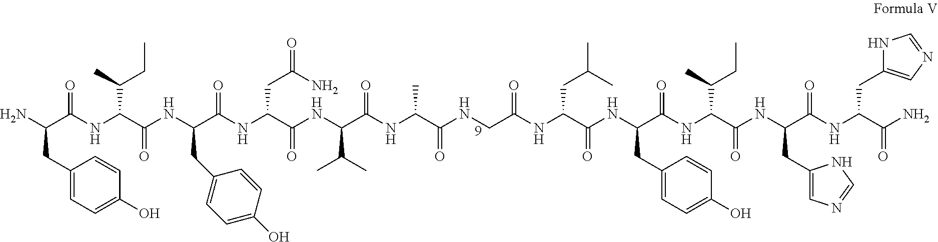 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

In some embodiments, provided is a peptide having the structure of Formula VI or a pharmaceutically acceptable salt thereof:

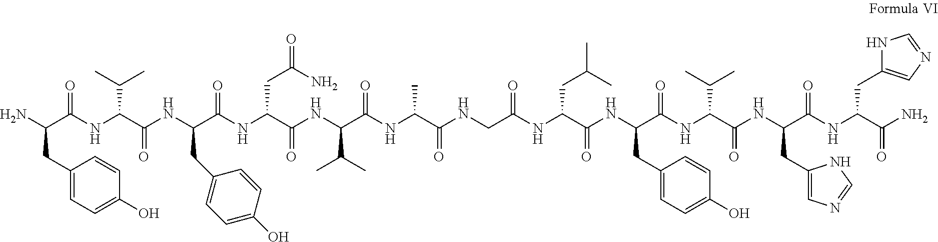 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

In some embodiments, provided is a peptide having the structure of Formula VII or a pharmaceutically acceptable salt thereof:

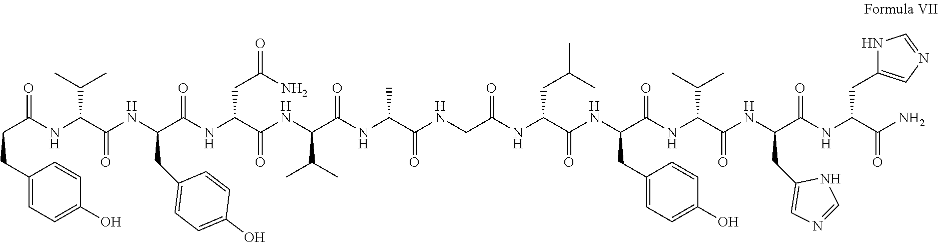

In some embodiments, provided is a peptide having the structure of Formula VIII or a pharmaceutically acceptable salt thereof:

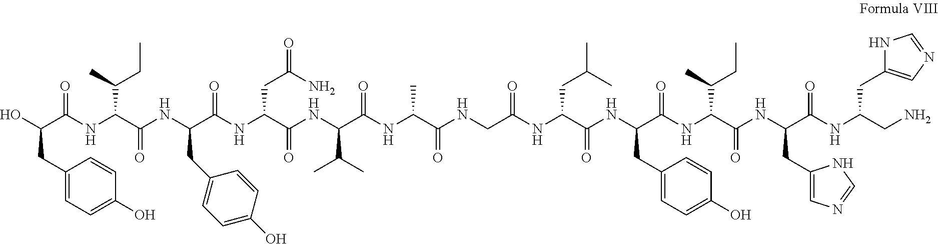 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@@H](CN)Cc1cnc[nH]1)[C@@H](C)CC)C(C)C]

In some embodiments, provided is a peptide having the structure of Formula IX or a pharmaceutically acceptable salt thereof:

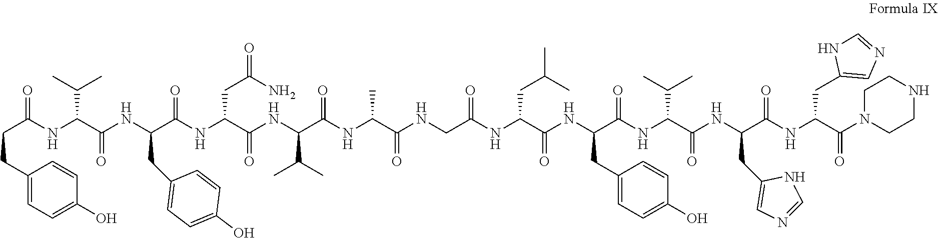

Further provided herein are salts of the peptide for inhibiting Fas-mediated inflammation in the eye and for use in the methods described here. As used herein, salt is generally synonymous with pharmaceutically acceptable salts, and/or includes or refers to pharmaceutically acceptable salts. Examples of pharmaceutically acceptable salts are salts with organic or inorganic acids such as (but not limited to) include acetic acid, aspartic acid, benzenesulfonic acid, benzoic acid, butyric acid, citric acid, fumaric acid, hydrochloric acid, hydrobromic acid, lactic acid, maleic acid, malonic acid, methanesulfonic acid, 4-methylbenzenesulfonic acid, nicotinic acid, phosphoric acid, succinic acid, sulfuric acid, or tartaric acid, prepared using methods well known in the art. In some embodiments, the salt is a hydrochloride salt.

In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, and polyamine resins.

Salts and pharmaceutically acceptable salts are described in in J. Pharmaceutical Sciences, 66: 1-19 (1977), the contents of which are incorporated by reference herein. In some embodiments, the salt is an acetate salt. In some embodiments, the acetate salt is a poly-acetate salt. In some embodiments, the poly-acetate salt is a tri-acetate salt.

In an embodiment, further provided are pharmaceutical compositions (also referred to as compositions) comprising the Fas inhibiting peptides. In some embodiments, the pharmaceutical compositions described herein comprise the peptide (e.g., a peptide comprising the amino acid sequence HHIYLGAVNYIY) or a pharmaceutically acceptable salt thereof. In some embodiments, the pharmaceutical compositions described herein comprise the peptide comprising the amino acid sequence HHIYLGAVNYIY or variant sequence thereof, or a pharmaceutically acceptable salt of the peptide. In some embodiments, the pharmaceutical compositions described herein comprise the peptide having the structure of any one of Formulas I-IX or a pharmaceutically acceptable salt thereof. In some embodiments, the pharmaceutical compositions described herein comprise the peptide having the structure of Formula I or a pharmaceutically acceptable salt thereof. In some embodiments, the pharmaceutical compositions described herein comprise the peptide having the structure of Formula III or a pharmaceutically acceptable salt thereof.

The pharmaceutical composition can comprise one or more excipients. As used herein, an excipient includes and/or refers to any pharmaceutically acceptable additive, carrier, diluent, adjuvant, or other ingredient, other than the active pharmaceutical ingredient (API), which is typically included for formulation and/or administration to a patient. A pharmaceutical composition can comprise a single pharmaceutical formulation (e.g., extended release, immediate release, delayed release, nanoparticulate, etc.) or multiple formulations (e.g., immediate release and delayed release, nanoparticulate and nonnanoparticulate, etc.). An excipient further includes and/or refers to an agent that may be added to a formulation to provide a desired consistency (e.g., altering the bulk properties), to improve stability, and/or to adjust osmolality. Examples of commonly used excipients include, but are not limited to, sugars, polyols, amino acids, surfactants, and polymers. In some embodiments, a non-ionic excipient or a non-ionizable excipient, as used herein, includes and/or refers to an agent having no net charge.

In some embodiments, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

In some embodiments, the compositions comprise excipients that are suitable for ocular application. Suitable excipients and include, but are not limited to, tonicity agents, preservatives, chelating agents, buffering agents, surfactants, cosolvents and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1, and the like. Suitable chelating agents include sodium edetate and the like. Suitable buffering agents include phosphates, borates, citrates, acetates, tromethamine, and the like. Suitable surfactants include ionic and nonionic surfactants. In some embodiments, the one or more excipients comprises nonionic surfactants, such as polysorbates, polyethoxylated castor oil derivatives, polyethoxylated fatty acids, polyethoxylated alcohols, polyoxyethylene-polyoxypropylene block copolymers (Poloxamer), and oxyethylated tertiary octylphenol formaldehyde polymer (Tyloxapol). Other suitable surfactants may also be included. Suitable antioxidants include sulfites, thiosulfate, ascorbates, BHA, BHT, tocopherols, and the like.

In some embodiments, the composition comprises a non-ionic surfactant. In some embodiments, the composition comprises a polysorbate, a polyethoxylated castor oil derivative, a polyethoxylated fatty acid, a polyethoxylated alcohol, a polyoxyethylene-polyoxypropylene block copolymer (Poloxamer), or an oxyethylated tertiary octylphenol formaldehyde polymer (Tyloxapol). In some embodiments, the composition comprises a polysorbate. In some embodiments, the composition comprises a polyethoxylated castor oil derivative. In some embodiments, the composition comprises a polyethoxylated fatty acid. In some embodiments, the composition comprises a polyethoxylated alcohol. In some embodiments, the composition comprises a polyoxyethylene-polyoxypropylene block copolymer (Poloxamer). In some embodiments, the composition comprises an oxyethylated tertiary octylphenol formaldehyde polymer (Tyloxapol). In some embodiments, the surfactant makes up 0.05%-20% weight per weight (w/w) of the composition. In some embodiments, the non-ionic surfactant is about 0.05% w/w of the composition to about 20% w/w of the composition. In some embodiments, the non-ionic surfactant is at least about 0.05% w/w of the composition. In some embodiments, the non-ionic surfactant is at most about 20% w/w of the composition. In some embodiments, the non-ionic surfactant is about 0.05% w/w of the composition to about 0.1% w/w of the composition, about 0.05% w/w of the composition to about 0.5% w/w of the composition, about 0.05% w/w of the composition to about 1% w/w of the composition, about 0.05% w/w of the composition to about 2% w/w of the composition, about 0.05% w/w of the composition to about 5% w/w of the composition, about 0.05% w/w of the composition to about 10% w/w of the composition, about 0.05% w/w of the composition to about 20% w/w of the composition, about 0.1% w/w of the composition to about 0.5% w/w of the composition, about 0.1% w/w of the composition to about 1% w/w of the composition, about 0.1% w/w of the composition to about 2% w/w of the composition, about 0.1% w/w of the composition to about 5% w/w of the composition, about 0.1% w/w of the composition to about 10% w/w of the composition, about 0.1% w/w of the composition to about 20% w/w of the composition, about 0.5% w/w of the composition to about 1% w/w of the composition, about 0.5% w/w of the composition to about 2% w/w of the composition, about 0.5% w/w of the composition to about 5% w/w of the composition, about 0.5% w/w of the composition to about 10% w/w of the composition, about 0.5% w/w of the composition to about 20% w/w of the composition, about 1% w/w of the composition to about 2% w/w of the composition, about 1% w/w of the composition to about 5% w/w of the composition, about 1% w/w of the composition to about 10% w/w of the composition, about 1% w/w of the composition to about 20% w/w of the composition, about 2% w/w of the composition to about 5% w/w of the composition, about 2% w/w of the composition to about 10% w/w of the composition, about 2% w/w of the composition to about 20% w/w of the composition, about 5% w/w of the composition to about 10% w/w of the composition, about 5% w/w of the composition to about 20% w/w of the composition, or about 10% w/w of the composition to about 20% w/w of the composition. In some embodiments, the non-ionic surfactant is about 0.05% w/w of the composition, about 0.1% w/w of the composition, about 0.5% w/w of the composition, about 1% w/w of the composition, about 2% w/w of the composition, about 5% w/w of the composition, about 10% w/w of the composition, or about 20% w/w of the composition.

In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the non-ionic surfactant comprises Polysorbate 20, Poloxamer 407, Tyloxapol, or cremophor. In some embodiments, the non-ionic surfactant is Polysorbate 20. In some embodiments, the non-ionic surfactant is Poloxamer 407. In some embodiments, the non-ionic surfactant is Tyloxapol. In some embodiments, the non-ionic surfactant is cremophor. The non-ionic surfactants described herein can be present within any one of the ranges (e.g., percent w/w) described herein, a specific value that falls within the described ranges.

In some embodiments, the composition further comprises cosolvents (e.g., between 0.5 and 50% w/w), such as N,N-Dimethylacetamide, ethanol, PEG-400, propylene glycol, dimethylsulfoxide (DMSO); oils, or cyclodextrins may be added to a pharmaceutical preparation. In some embodiments, the composition further comprises a tonicity-adjusting agent. In some embodiments, the composition is an isotonic solution. In some embodiments, the tonicity-adjusting agent is mannitol, sorbitol, glucose or trehalose, or an inorganic salt such as sodium chloride. In some embodiments, the composition comprises mannitol. In some embodiments, the composition comprises sorbitol. In some embodiments, the composition comprises glucose or trehalose. In some embodiments, the composition comprises an inorganic salt. In some embodiments, the tonicity-adjusting agent is present at an amount suitable to bring the tonicity of the composition into the 250-400 mOsm/L range. In some embodiments, the non-ionic surfactant is about 1% w/w of the composition to about 10% w/w of the composition. In some embodiments, the non-ionic surfactant is at least about 1% w/w of the composition. In some embodiments, the non-ionic surfactant is at most about 10% w/w of the composition. In some embodiments, the non-ionic surfactant is about 1% w/w of the composition to about 2% w/w of the composition, about 1% w/w of the composition to about 3% w/w of the composition, about 1% w/w of the composition to about 4% w/w of the composition, about 1% w/w of the composition to about 5% w/w of the composition, about 1% w/w of the composition to about 10% w/w of the composition, about 2% w/w of the composition to about 3% w/w of the composition, about 2% w/w of the composition to about 4% w/w of the composition, about 2% w/w of the composition to about 5% w/w of the composition, about 2% w/w of the composition to about 10% w/w of the composition, about 3% w/w of the composition to about 4% w/w of the composition, about 3% w/w of the composition to about 5% w/w of the composition, about 3% w/w of the composition to about 10% w/w of the composition, about 4% w/w of the composition to about 5% w/w of the composition, about 4% w/w of the composition to about 10% w/w of the composition, or about 5% w/w of the composition to about 10% w/w of the composition. In some embodiments, the non-ionic surfactant is about 1% w/w of the composition, about 2% w/w of the composition, about 3% w/w of the composition, about 4% w/w of the composition, about 5% w/w of the composition, or about 10% w/w of the composition.

In some embodiments, the composition comprises a buffering agent. In some embodiments, the buffering agent is an acidifying agent. In some embodiments, the acidifying agent is an acetate buffer at pH 4.5. In some embodiments, the concentration acetate buffer pH 4.5 is about 10 millimolar (mM). Generally, the pH may be controlled by an appropriate buffer suitable for injection into the eye, for example the pH of the composition can be in the 3.0-7.5 range or 3.5-4.5 range.

As described herein, the composition can comprise one or more excipients. Accordingly, in some embodiments, the composition comprises a non-ionic surfactant, a tonicity-adjusting agent, and a buffering agent, in combination with the peptide. Any of the described excipients can be combined within the amounts and/or ranges described.

In an embodiment, the compositions described herein comprise an amount of the peptide suitable to inhibit Fas-mediated inflammation and/or treat, inhibit and/or reduce inherited retinal degeneration or retinal cell death or the symptoms thereof in an eye. As used herein, a dose or dosage includes and/or refers to the amount of therapeutic agent, such as the peptides described, in a composition (e.g., a composition for administering to an eye). A dose can refer to either (i) the peptide (parent compound) or the pharmaceutically acceptable salt thereof. In some embodiments, the amount of the peptide in the composition (i.e., pharmaceutical composition) that is suitable for the methods described herein (e.g., treating retinal lesions) ranges from 5 micrograms (ug) to 10,000 ug. The dosing forms comprising the compositions described herein are generally administered to the vitreous humor of an eye and can be further formulated for injection into the eye (e.g., intravitreal injection). In some embodiments, the amount of the peptide (e.g., a peptide comprising the amino acid sequence HHIYLGAVNYIY) or a pharmaceutically acceptable salt thereof that is suitable for the methods described herein ranges from 5 ug to 10,000 ug. In some embodiments, the amount of the peptide comprising the amino acid sequence HHIYLGAVNYIY or variant sequence thereof, or a pharmaceutically acceptable salt of the peptide that is suitable for the methods described herein ranges from 5 ug to 10,000 ug. In some embodiments, the amount of the peptide having the structure of Formula I or a pharmaceutically acceptable salt thereof that is suitable for the methods described herein ranges from 5 ug to 10,000 ug. In some embodiments, the amount of the peptide having the structure of Formula III or a pharmaceutically acceptable salt thereof that is suitable for the methods described herein ranges from 5 ug to 10,000 ug.

In some embodiments, a dose comprises about 5-1,000 ug of the peptide (e.g., Formula III) or the variant sequence thereof. In some embodiments, a dose comprises about 25-500 ug of the peptide or the variant sequence thereof. In some embodiments, a dose comprises about 25-250 ug of the peptide or the variant sequence thereof. In some embodiments, a dose comprises about 50-250 ug of the peptide or the variant sequence thereof. In certain embodiments, a dose comprises about 50 ug of the peptide or the variant sequence thereof. In certain embodiments, a dose comprises about 100 ug of the peptide or the variant sequence thereof. In certain embodiments, a dose comprises about 200 ug of the peptide or the variant sequence thereof. In certain embodiments, a dose comprises about 300 ug of the peptide or the variant sequence thereof. In some embodiments, the peptide is present at a concentration 0.1 milligrams per milliliter (mg/ml) to 10 mg/ml. In some embodiments, the peptide is present at a concentration 0.1 milligrams per milliliter (mg/ml) to 5.0 mg/mL.

In some embodiments, a dose comprises about 5 ug of a pharmaceutically acceptable salt of the peptide to about 300 ug of a pharmaceutically acceptable salt of the peptide. In some embodiments, a dose comprises at least about 5 ug of a pharmaceutically acceptable salt of the peptide. In some embodiments, a dose comprises at most about 300 ug of a pharmaceutically acceptable salt of the peptide.

The concentration of the peptide within the composition can be adjusted in a manner suitable for ocular administration. In some embodiments, the concentration of the peptide within the composition ranges from about 0.1 milligrams per milliliter (mg/ml) to about 5 mg/ml. In some embodiments, the concentration of the peptide within the composition ranges from about 0.1 milligrams per milliliter (mg/ml) to about 10 mg/ml. In some embodiments, the concentration of the peptide within the composition ranges from about 0.1 milligrams per milliliter (mg/mL) to about 100 mg/ml. In some embodiments, the concentration of the peptide (e.g., a peptide comprising the amino acid sequence HHIYLGAVNYIY) or a pharmaceutically acceptable salt thereof ranges from about 0.1 mg/ml to about 5 mg/mL. In some embodiments, the concentration of the peptide comprising the amino acid sequence HHIYLGAVNYIY or variant sequence thereof, or a pharmaceutically acceptable salt of the peptide ranges from about 0.1 milligrams per milliliter (mg/ml) to about 5 mg/ml. In some embodiments, the concentration of the peptide having the structure of Formula I or a pharmaceutically acceptable salt thereof ranges from about 0.1 mg/ml to about 5 mg/mL. In some embodiments, the concentration of the peptide having the structure of Formula III or a pharmaceutically acceptable salt thereof ranges from about 0.1 mg/ml to about 5 mg/mL.

In some embodiments, the compositions described herein are administered to an eye of an individual in need thereof. Administration to an eye (i.e., “ocular application” or “ocular administration”) includes subconjunctival, intravitreal, retrobulbar, intracameral administration subretinal, or suprachoroidal. In some embodiments, ocular administration comprises subconjunctival, intravitreal, retrobulbar, or intracameral administration. In some embodiments, ocular administration comprises intravitreal administration. In some embodiments, ocular administration comprises subconjunctival administration. In some embodiments, ocular administration comprises retrobulbar administration. In some embodiments, ocular administration comprises intracameral administration.

In some embodiments, the dosing forms comprising the compositions described herein are generally administered to the vitreous humor of an eye. In some embodiments, the half-life of the peptide (e.g., a peptide comprising the amino acid sequence HHIYLGAVNYIY) or a pharmaceutically acceptable salt thereof in the vitreous humor is greater than about 30 days to greater than about 275 days. In some embodiments, the peptide comprising the amino acid sequence HHIYLGAVNYIY or variant sequence thereof, or a pharmaceutically acceptable salt of the peptide has a half-life in the vitreous humor that is greater than about 30 days to greater than about 275 days. In some embodiments, the peptide having the structure of Formula I or a pharmaceutically acceptable salt thereof has a half-life in the vitreous humor that is greater than is greater than about 30 days to greater than about 275 days. In some embodiments, the peptide having the structure of Formula III or a pharmaceutically acceptable salt thereof has a half-life in the vitreous humor that is greater than is greater than about 30 days to greater than about 275 days. In some embodiments, the peptide comprising the amino acid sequence HHIYLGAVNYIY or variant sequence thereof, or a pharmaceutically acceptable salt of the peptide has a half-life in the vitreous humor that is greater than about 14 days to greater than about 275 days. In some embodiments, the peptide having the structure of Formula I or a pharmaceutically acceptable salt thereof has a half-life in the vitreous humor that is greater than is greater than about 14 days to greater than about 275 days. In some embodiments, the peptide having the structure of Formula III or a pharmaceutically acceptable salt thereof has a half-life in the vitreous humor that is greater than is greater than about 14 days to greater than about 275 days.

In some embodiments, the half-life of the peptide is greater than about 14 days in the eye. In some embodiments, the half-life of the peptide is greater than about 30 days in the eye. In some embodiments, the half-life of the peptide is greater than about 60 days in the eye. In some embodiments, the half-life of the peptide is greater than about 90 days in the eye. In some embodiments, the half-life of the peptide is greater than about 120 days in the eye. In some embodiments, the half-life of the peptide is greater than about 150 days in the eye. In some embodiments, the half-life of the peptide is greater than about 180 days in the eye. In some embodiments, the half-life of the peptide is greater than about 210 days in the eye. In some embodiments, the half-life of the peptide is greater than about 240 days in the eye. In some embodiments, the half-life of the peptide is greater than about 270 days in the eye.

In some embodiments, the half-life of the peptide is greater than about 14 days in the vitreous humor. In some embodiments, the half-life of the peptide is greater than about 30 days in the vitreous humor. In some embodiments, the half-life of the peptide is greater than about 60 days in the vitreous humor. In some embodiments, the half-life of the peptide is greater than about 90 days in the vitreous humor. In some embodiments, the half-life of the peptide is greater than about 120 days in the vitreous humor. In some embodiments, the half-life of the peptide is greater than about 150 days in the vitreous humor. In some embodiments, the half-life of the peptide is greater than about 180 days in the vitreous humor. In some embodiments, the half-life of the peptide is greater than about 210 days in the vitreous humor. In some embodiments, the half-life of the peptide is greater than about 240 days in the vitreous humor. In some embodiments, the half-life of the peptide is greater than about 270 days in the vitreous humor.

Determining the amount of the peptide in the vitreous humor generally requires collecting all of the vitreous fluid or a substantial portion thereof from an eye or sacrificing the eye in order to sample the vitreous humor. In some embodiments, collecting all of the vitreous fluid or a substantial portion thereof in a human eye, or sacrificing an eye is not feasible for maintaining the health of an eye in a human. Accordingly, in some embodiments, the half-life of the peptide in a human eye is determined by measuring and/or extrapolating from a half-life of the peptide in the eye of a mammal. In some embodiments, the mammal is a rabbit. In some embodiments, the mammal is a pig (e.g., minipig). In some embodiments, the mammal is a monkey. Various methods of detecting the presence of a drug are also suitable for detecting the peptide. For example, methods suitable for detecting the peptide include performing mass spectrometry (e.g., liquid chromatography-mass spectrometry (LC-MS) or high-performance LC-MS (HPLC-MS)) on a sample from the vitreous humor.

Provided and described herein are compositions and methods useful for treating inherited retinal degeneration or retinal cell death. In some embodiments, provided and exemplified herein are methods of treating inherited retinal degeneration in an eye of an individual, wherein the method comprises: administering: (i) an autophagy inhibitor to the individual; and (ii) a Fas-inhibiting peptide to the eye, wherein the peptide comprises an amino acid sequence HHIYLGAVNYIY or variant sequence thereof, or a pharmaceutically acceptable salt thereof. In certain embodiments, inherited retinal degeneration comprises retinitis pigmentosa. In certain embodiments, the individual has a P23H opsin mutation.

In some embodiments, provided and exemplified herein are methods of treating retinal cell death in an eye of an individual, wherein the method comprises: administering: (i) an autophagy inhibitor to the individual; and (ii) a Fas-inhibiting peptide to the eye, wherein the peptide comprises an amino acid sequence HHIYLGAVNYIY or variant sequence thereof, or a pharmaceutically acceptable salt thereof. In certain embodiments, the retinal cell death comprises photoreceptor cell death, retinal epithelium cell death, or both photoreceptor cell death and retinal epithelium cell death.

In certain embodiments, treating inherited retinal degeneration or retinal cell death comprises neuroprotection of photoreceptors (e.g., compared to an individual treated with hydroxychloroquine alone or to an individual treated with a Fas inhibitor alone). In certain embodiments, neuroprotection of photoreceptors comprises reducing a decline in photoreceptor loss (e.g., rate of loss), reducing a decline in outer nuclear layer thickness (e.g., the rate of outer nuclear layer thinning), reducing a decline in retinal nerve fiber layer thickness (e.g., the rate of nerve fiber layer thinning). In certain embodiments, treating inherited retinal degeneration or retinal cell death comprises reducing an amount of inflammatory cytokines in the eye (e.g., compared to an individual treated with hydroxychloroquine alone). In certain embodiments, reducing an amount of inflammatory cytokines in the eye is measured from a vitreous fluid sample. In certain embodiments, treating inherited retinal degeneration or retinal cell death comprises reducing a decline in outer nuclear layer thickness (e.g., the rate of outer nuclear layer thinning compared to an individual treated with hydroxychloroquine alone).

In certain embodiments, treating inherited retinal degeneration or retinal cell death comprises reducing a decline in retinal nerve fiber layer thickness (e.g., the rate of retinal nerve fiber layer thinning compared to an individual treated with hydroxychloroquine alone).

In certain embodiments, treating inherited retinal degeneration or retinal cell death comprises reducing a decline in visual function (e.g., light responsiveness) in the eye (e.g., the rate of responsiveness compared to an individual treated with hydroxychloroquine alone). In certain embodiments, treating inherited retinal degeneration or retinal cell death comprises decreasing a decline (e.g., rate) in retinal nerve fiber layer thickness.

In certain embodiments, a method of treating inherited retinal degeneration or retinal cell death provided herein comprises administering the peptide prior to the autophagy inhibitor. In certain embodiments, a method of treating inherited retinal degeneration or retinal cell death comprises administering the autophagy inhibitor prior to the peptide.

In certain embodiments, the autophagy inhibitor is hydroxychloroquine. In certain embodiments, the hydroxychloroquine comprises a hydroxychloroquine molecule or a salt of hydroxychloroquine, a metabolite of hydroxychloroquine, or prodrug of hydroxychloroquine. In certain embodiments, hydroxychloroquine is administered orally.

In some embodiments, the method comprises administering each composition to the vitreous humor of the eye. In some embodiments, the peptide has a half-life in the vitreous humor greater than about 30 days. In some embodiments, the peptide has a half-life in the vitreous humor greater than about 90 days. In some embodiments, the peptide has a half-life in the vitreous humor greater than about 180 days. In some embodiments, the peptide has a half-life in the vitreous humor greater than about 200 days. In some embodiments, the method comprises using the vitreous humor as a depot to provide the peptide to retinal tissue in the eye. In certain embodiments, the peptide is present in the vitreous humor greater than about 8 weeks after administration. In certain embodiments, the peptide is present in the vitreous humor greater than about 10 weeks after administration. In certain embodiments, the peptide is present in the vitreous humor greater than about 12 weeks after administration. In certain embodiments, the peptide is present in the vitreous humor greater than about 16 weeks after administration. In certain embodiments, the peptide is present in the vitreous humor greater than about 20 weeks after administration. In certain embodiments, the peptide is present in the vitreous humor greater than about 24 weeks after administration. In certain embodiments, the peptide is present in the vitreous humor greater than about 30 weeks after administration. In certain embodiments, the peptide is present in the vitreous humor greater than about 40 weeks after administration. In certain embodiments, the peptide is present in the vitreous humor greater than about 50 weeks after administration.

In some embodiments, treating a loss visual function in an eye having a lesion comprises reducing a loss (e.g., rate of loss) in visual function as compared to a baseline visual function prior to administering the peptide or the variant sequence thereof, or the pharmaceutically acceptable salt of the peptide. In some embodiments, treating a loss visual function in an eye having a lesion comprises increasing and/or improving visual function (e.g., improving BCVA) as compared to a baseline visual function prior to administering the peptide or the variant sequence thereof, or the pharmaceutically acceptable salt of the peptide. In some embodiments, the methods comprise increasing and/or improving visual function (e.g., improving BCVA) as compared to a baseline visual function prior to administering the peptide or the variant sequence thereof, or the pharmaceutically acceptable salt of the peptide. In some embodiments, visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, improving visual function comprises improving one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, visual function comprises best corrected visual acuity (e.g., increase in visual acuity from 20/200 to 20/100, or an increase in visual acuity from 20/50 to 20/40, and/or an increase in visual acuity from measured in a reduction in logMAR, etc.). As used herein, the term “best corrected visual acuity” or “BCVA”, generally refers to the minimum angle of resolution subtended by a certain number of arc minutes. In certain instances, the subtended visual angle of an object is the angle formed by rays projecting from the eye to the top and bottom (or left and right sides) of an object. In such instances, such visual angles are used to indicate the size of the retinal image of the object (e.g., the larger the visual angle, the larger the retinal image size is). In certain instances, the visual angle is influenced by two parameters: the size of the object and the distance of the object from the eye. Bigger objects cast larger images on the retina than smaller objects. Thus, the larger the object is, the larger its visual angle will be. Closer objects cast larger images on the retina than smaller objects. Thus, the closer the object is to the eye, the larger its visual angle will be. By way of example, standard vision is thus the ability to distinguish features separated by 1 minute of arc. By way of further example, the ability to distinguish a set of bars separated by 1 arc minute is 20/20 vision, 2 arc minutes is 20/40 vision. In some embodiments, visual acuity is measured by a Snellen chart, ETDRS chart, Landolt C chart, Tumbling E chart, HOTV chart, and/or logMAR (log minimum angle of resolution). In some embodiments, visual function comprises field of vision. In some embodiments, visual function comprises contrast sensitivity. In some embodiments, visual function comprises binocular function. In some embodiments, visual function comprises low luminance acuity. In some embodiments, visual function comprises low contrast acuity. In some embodiments, visual function comprises color vision. In some embodiments, visual function comprises perimetry. In some embodiments, visual function comprises threshold sensitivity. In some embodiment, visual function comprises reading speed. In some embodiments, visual function comprises light/dark adaptation. In some embodiments, treating visual function comprises reducing a loss in visual function as compared to a baseline visual function prior to the administering peptide or the variant sequence thereof, or the pharmaceutically acceptable salt of the peptide.

In some embodiments, the methods described herein include methods of treating vision loss associated with inflammation (e.g., Fas-mediated inflammation) in an eye. In some embodiments, retinal inflammation can be determined by observing the symptoms associated with inflammation in the eye (e.g., loss of and/or decrease in visual acuity, central vision loss, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-1b, IP-10, IL-18, MIP-1a, IL-6, GFAP, MIP2, MCP-1, or MIP-1b); a Fas-mediated complement-related molecules (complement component 3 (C3) or complement component 1 q (C1q)) Caspase 8; components of the inflammasome (e.g., NLRP3 or NLRP2); C-X-C motif chemokines (e.g., CXCL2 (MIP-2alpha) or CXCL10 (IP-10)); C-X3-C motif chemokines (e.g., CX3CL1 (fractalkine)); C-C motif chemokines (CCL2 (MCP-1), CCL3 (MIP-1a), and CCL4 (MIP-1b)); toll-like receptor 4 (TLR4); interleukin cytokines (e.g., IL-1b, IL-18, and IL-6); TNF superfamily cytokines (e.g., TNFa); or GFAP.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions. In some embodiments, the variant sequence comprises a truncation.

In some embodiments, the peptide further comprises a modification. In some embodiments, comprises a modified amino acid or a non-natural amino acid. In some embodiments, the peptide comprises an amidated C-terminus. In some embodiments, the peptide has the structure of Formula I, or a pharmaceutically acceptable salt thereof. In some embodiments, the peptide has the structure of Formula III, or a pharmaceutically acceptable salt thereof.

In some embodiments, the pharmaceutically acceptable salt of the peptide is administered. In some embodiments, the pharmaceutically acceptable salt is an acetate salt. In some embodiments, the pharmaceutically acceptable salt is a polyacetate salt. In some embodiments, the polyacetate salt is a triacetate salt. In some embodiments, the pharmaceutically acceptable salt is a hydrochloride salt.

In some embodiments, the peptide or the variant sequence thereof, or the pharmaceutically acceptable salt of the peptide is formulated in a composition (e.g., the pharmaceutical compositions described herein).

In some embodiments, about 5-1,000 ug of the peptide or the variant sequence thereof, or the pharmaceutically acceptable salt of the peptide is administered. In some embodiments, about 25-500 ug of the peptide or the variant sequence thereof, or the pharmaceutically acceptable salt of the peptide is administered. In some embodiments, about 25-250 ug of the peptide or the variant sequence thereof, or the pharmaceutically acceptable salt of the peptide is administered. In some embodiments, about 50-250 ug of the peptide or the variant sequence thereof, or the pharmaceutically acceptable salt of the peptide is administered. In certain embodiments, about 50 ug of the peptide or the variant sequence thereof, or the pharmaceutically acceptable salt of the peptide is administered. In certain embodiments, about 100 ug of the peptide or the variant sequence thereof, or the pharmaceutically acceptable salt of the peptide is administered. In certain embodiments, about 200 ug of the peptide or the variant sequence thereof, or the pharmaceutically acceptable salt of the peptide is administered. In some embodiments, the peptide is present at a concentration 0.1 milligrams per milliliter (mg/ml) to 10 mg/mL. In some embodiments, the peptide is present at a concentration 0.1 milligrams per milliliter (mg/ml) to 5.0 mg/mL.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, the term “inhibition” or “inhibiting” or reducing includes and/or refers to the reduction or suppression of a given condition, symptom, disorder, or disease, and/or a decrease in the baseline activity of a biological activity or process.

As used herein, the term “treating” or “treatment” of includes and/or refers to ameliorating the disease or disorder or symptoms thereof (e.g., slowing or arresting or reducing the development of the disease or at least one of the clinical symptoms thereof). In certain embodiments, “treating” or “treatment” also includes and/or refers to alleviating or ameliorating at least one physical and/or biological parameters including those which may not be discernible by the patient. In certain embodiments, “treating” or “treatment” includes and/or refers to modulating a disease, disorder, or biological process either physically, (e.g., stabilization of a discernible symptom), physiologically, (e.g., stabilization of a physical and/or biological parameter), or both. In certain embodiments, “treating” or “treatment” includes and/or refers to preventing or delaying the onset or development or progression of the disease or disorder. In certain embodiments, “treating” or “treatment” includes and/or refers to preventing or delaying or inhibiting the deterioration of (i) a healthy physiological state or (ii) a baseline physiological state (e.g., the progression of a disease or disorder).

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

As used herein, in any instance or embodiment described herein, “comprising” may be replaced with “consisting essentially of” and/or “consisting of”. Used herein, in any instance or embodiment described herein, “comprises” may be replaced with “consists essentially of” and/or “consists of”.

### EXAMPLES

### Example 1—Protection of Photoreceptor Survival Using an Autophagy Inhibitor, Hydroxychloroquine (HCQ), in Drinking Water in a Fas-Deficient Mouse Model of Retinitis Pigmentosa (Fas-Lpr/P23H).

The purpose of this study is to examine whether inhibition of autophagy, via administration of an autophagy inhibitor, and inhibition of Fas pathways, via a Fas-deficient mouse model, protects photoreceptor survival in a mouse model of retinitis pigmentosa (Fas-Lpr/P23H.)

### Materials and Methods

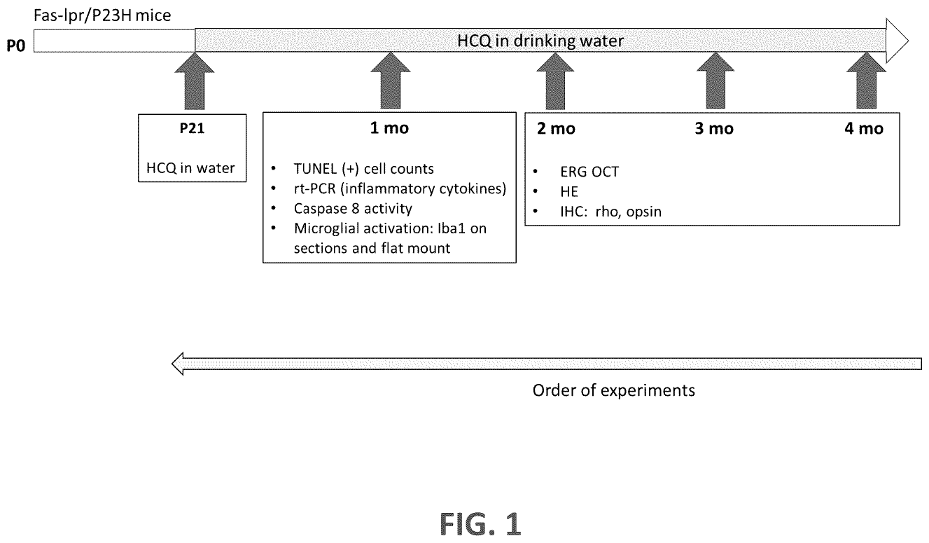

This study was conducted in the Lpr/P23H mouse line, a Fas-deficient mouse line that is also a model of retinitis pigmentosa. Hydroxychloroquine (HCQ) was given in the drinking water at 1.2 mg/ml starting at 21 days post-natal (P21) to reduce autophagy flux (FIG. 1). Photoreceptor death was assessed by Terminal deoxynucleotidyl transferase (TdT) dUTP Nick-End Labeling (TUNEL) cell counts. Retinal structure was examined by histology and optical coherence tomography (OCT) analysis. Electroretinogram (ERG) recording and immunohistochemistry (IHC) staining of rhodopsin and m-opsin were performed to evaluate the retinal function. Transcript levels of inflammatory cytokines, CCL-2 and CCL-3, were analyzed by rt-PCR.

### Results

Treatment with autophagy inhibitor HCQ preserved photoreceptor survival and function and lowered the production of inflammatory cytokines in the retinas of the a Fas-deficient mouse model of retinitis pigmentosa, Fas-Lpr/P23H mice.

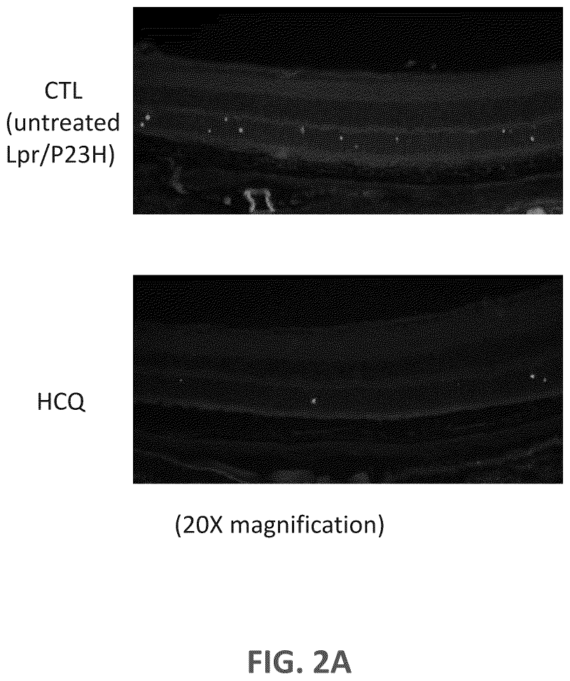

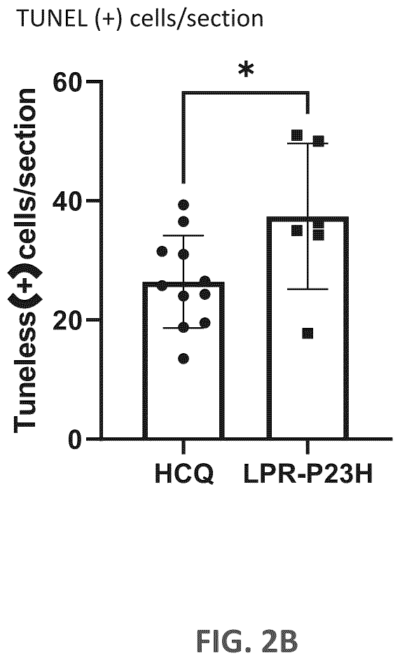

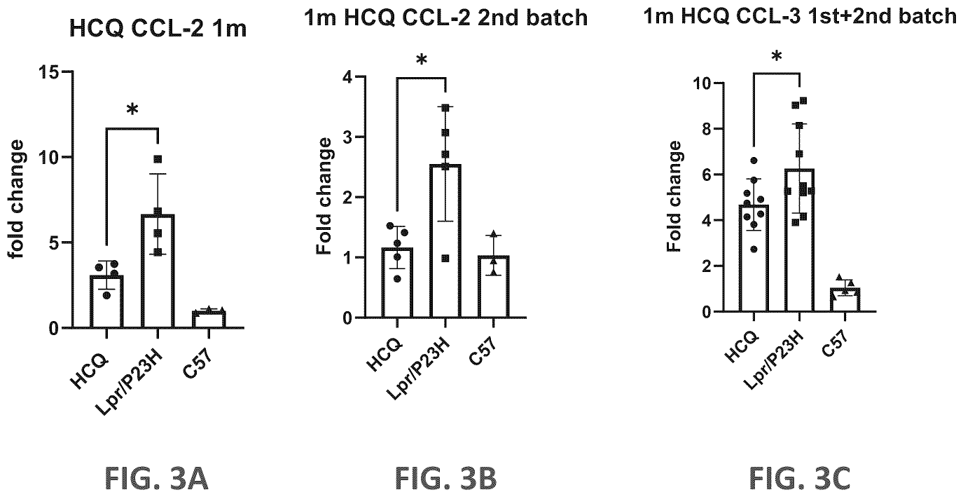

Photoreceptor survival was assessed using TUNEL cell counts in Fas-Lpr/P23H mice that were administered the autophagy inhibitor HCQ and compared to TUNEL cell counts in mice that were not administered HCQ (untreated). Example images of the retina of a mouse that was untreated (top) and a retina of a mouse that received HCQ (bottom) are provided in FIG. 2A. In these example images, there are fewer TUNEL-positive cells in Fas-Lpr/P23H mice that received HCQ than there are in mice that did not receive HCQ. FIG. 2B provides TUNEL-positive cell counts in the retinas of Fas-Lpr/P23H mice that received HCQ and that were untreated. There was statistically significantly fewer TUNEL-positive cells in mice that were administered HCQ than in mice that did not receive HCQ. Together, these results indicate that there was less cell death in mice that received an autophagy inhibitor than in mice that did not. Additionally, HCQ treatment lowered production of inflammatory cytokine CCL-2 (FIG. 3A and FIG. 3B) and of inflammatory cytokine CCL-3 (FIG. 3C) in mice that received HCQ in comparison to mice that did not receive HCQ, indicating that the administration of HCQ decreases retinal cell inflammation.

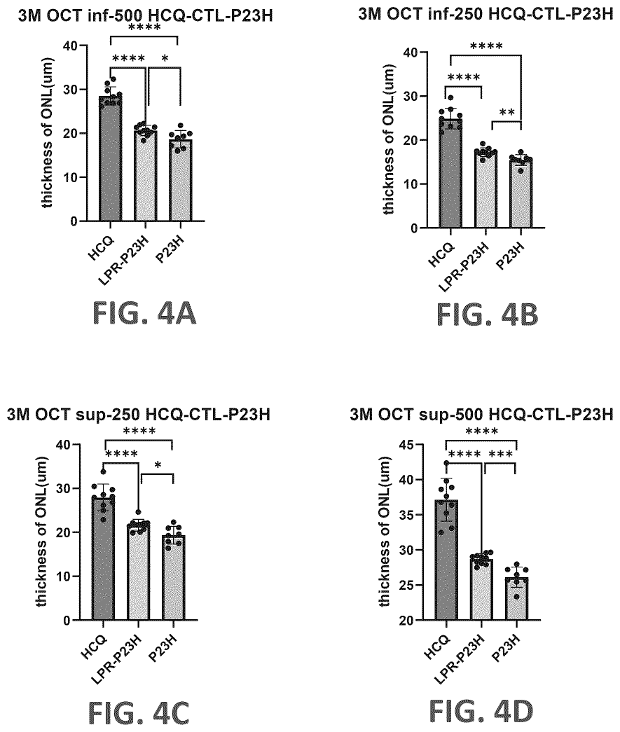

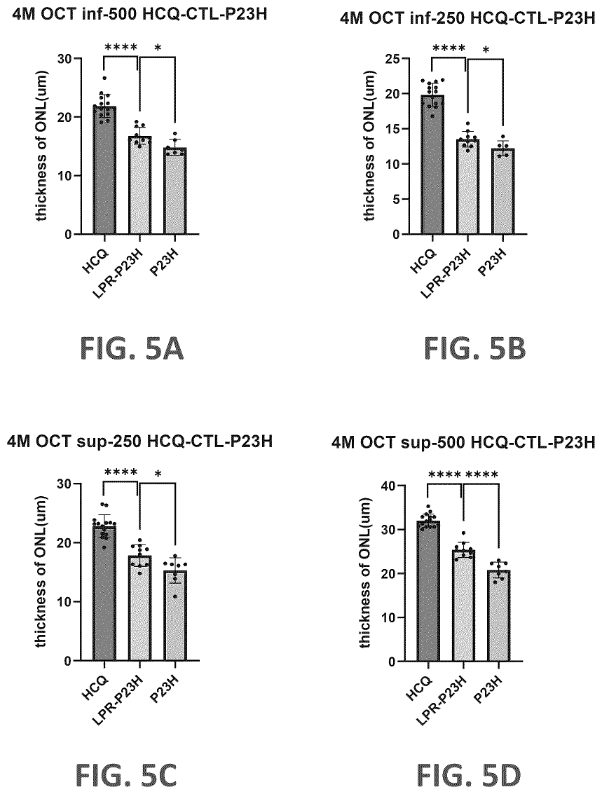

A proxy for retinal cell death is optic nerve layer (ONL) thickness, with thicker ONLs correlating to less cell death. ONL thickness was measured at 250 and 500 μm from the optic nerve in the inferior and the superior areas of the retina in mice that were 3-months old (inferior FIG. 4A and FIG. 4B; superior FIG. 4C and FIG. 4D) and in mice that were 4-months old (inferior FIG. 5A and FIG. 5B; superior FIG. 5C and FIG. 5D). Fas-deficient Lpr/P23H mice exhibited significantly greater ONL thickness compared with P23H mice that were not deficient. Moreover, Fas-deficient LPR/P23H mice that were administered HCQ had significantly greater ONL thickness than did Fas-Lpr/P23H mice that were not administered HCQ. These data indicate that the combination of a Fas inhibitor and an autophagy inhibitor provides effective protection to retinal cells.

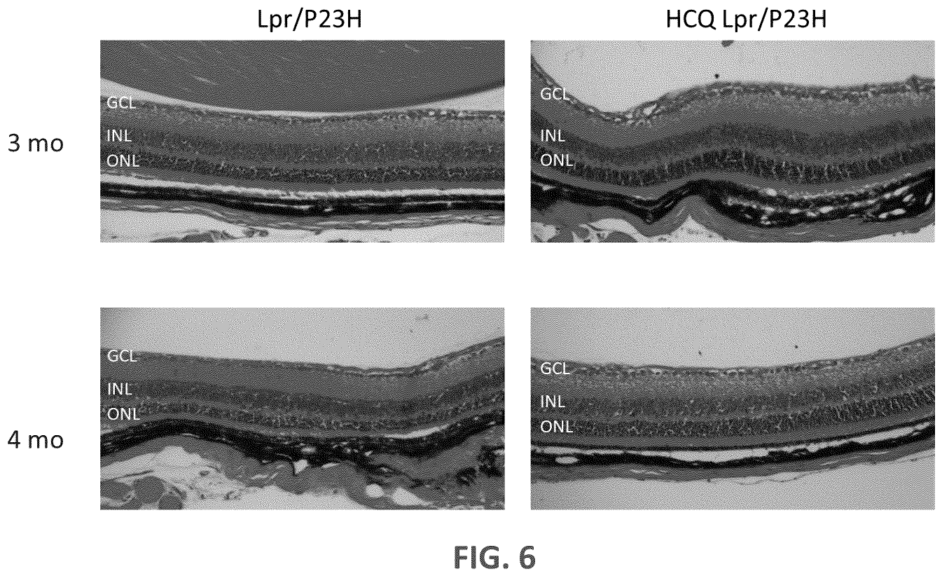

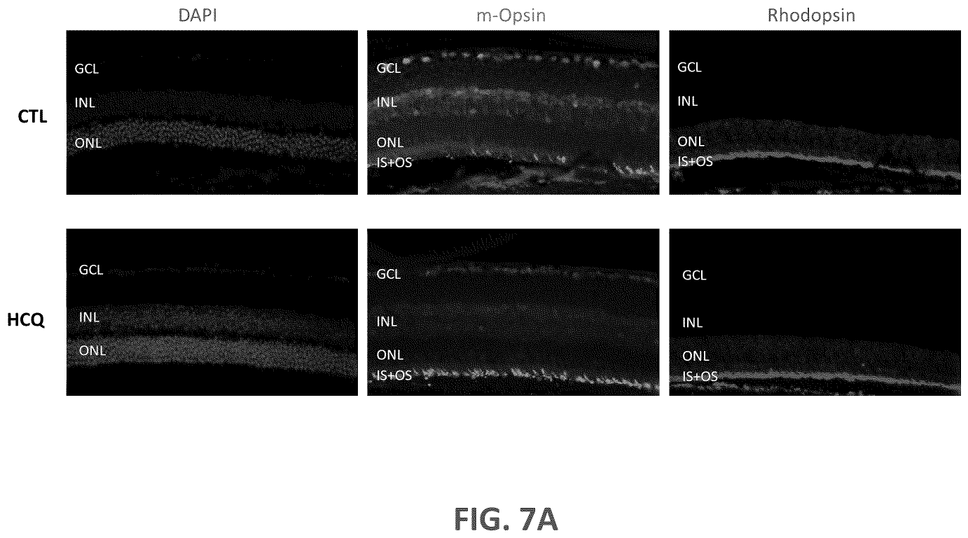

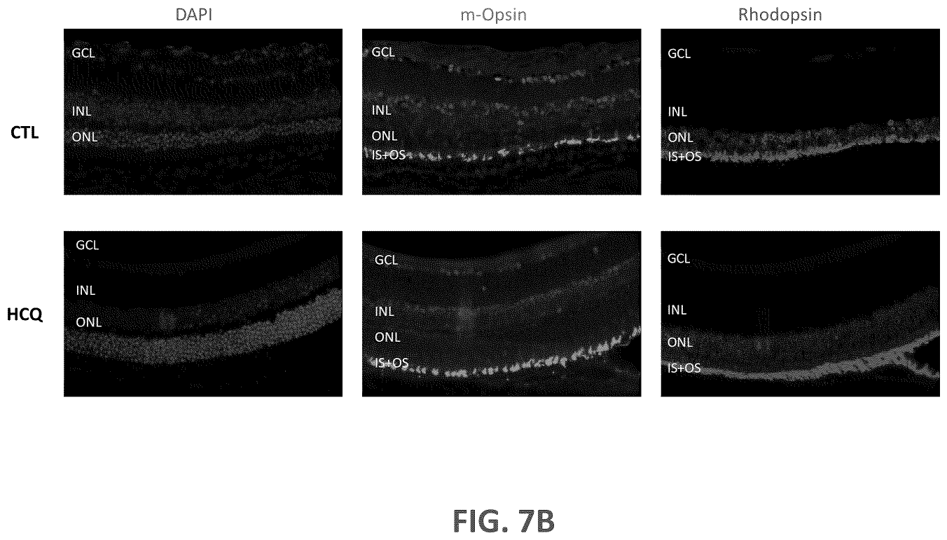

Example histological images of 3-month and 4-month old mouse retinas show greater photoreceptor survival in Fas-LPR/P23H mice that received HCQ than in mice that did not (FIG. 6). Additionally, IHC images staining for cell bodies (DAPI), cones (m-opsin), and rods (rhodopsin) show greater survival of cell bodies, cones, and rods in both 3-month (FIG. 7A) and 4-month (FIG. 7B) old Fas-Lpr/P23H in mice that received HCQ (bottom row) than in mice that did not (top row.)

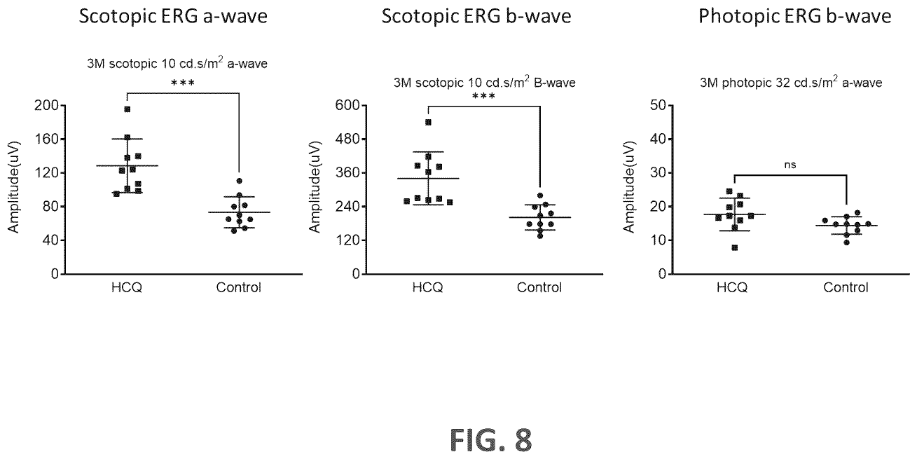

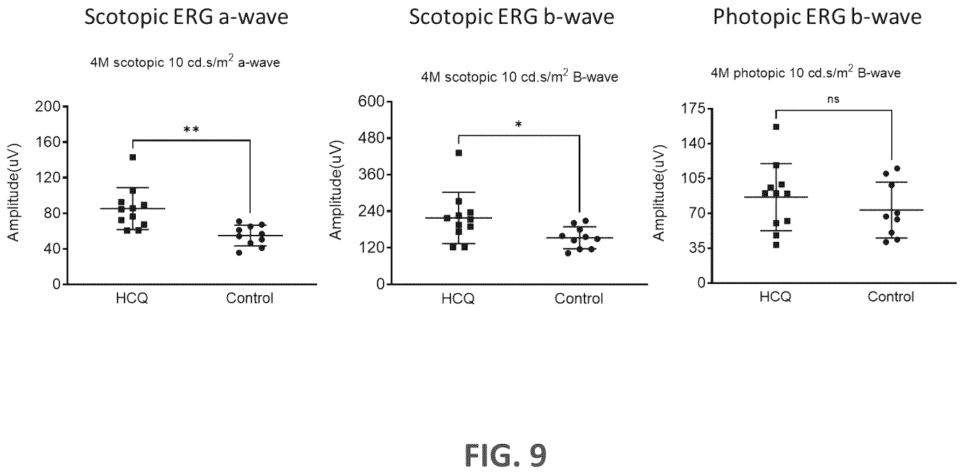

Photoreceptor function was assessed using electroretinograms (ERGs). In both 3-month (FIGS. 8) and 4-month (FIG. 9) old Fas-LPR/P23H mice scotopic ERG a-waves and b-waves had significantly greater amplitude in mice that received HCQ than in untreated mice, indicating that autophagy inhibition in combination with Fas inhibition preserved photoreceptor function.

### Example 2—Protection of Photoreceptor Survival Using a Combination of a Fas Inhibitor, ONL1204, Intravitreal Injections and an Autophagy Inhibitor, HCQ, in Drinking Water in a Mouse Model of Retinitis Pigmentosa (P23H).

The purpose of this study is to examine whether inhibition of autophagy, via administration of an autophagy inhibitor, and inhibition of Fas pathways, via intravitreal injections of a Fas inhibitor, protects photoreceptor survival in a mouse model of retinitis pigmentosa (P23H.)

### Materials and Methods

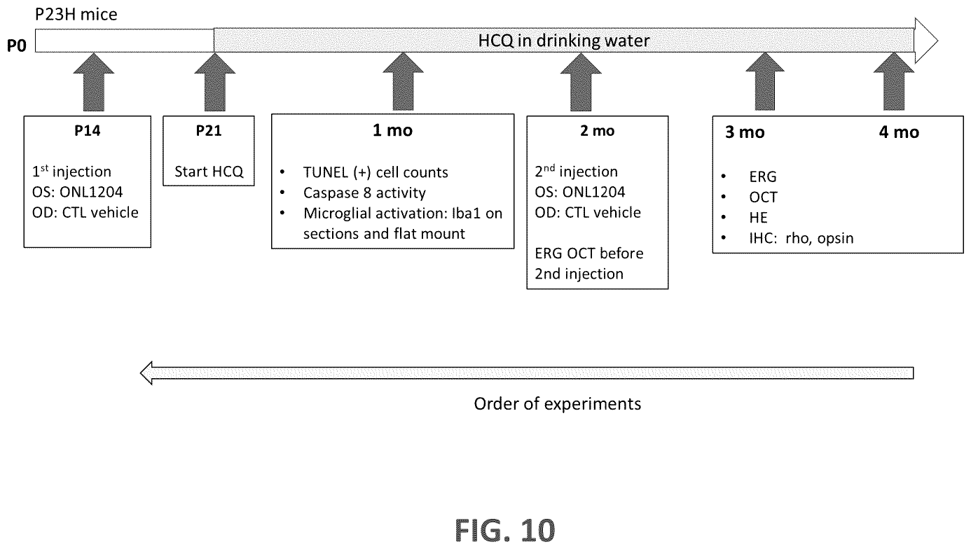

This study was conducted in the P23H mouse line, a model of retinitis pigmentosa. Hydroxychloroquine (HCQ) was given in the drinking water at 1.2 mg/ml starting at 21 days post-natal (P21) to reduce autophagy flux (FIG. 10). A Fas inhibitor, ONL1204, was administered via intravitreal injection at post-natal day 14 (P14) and 2 months of age. Retinal structure was examined by optical coherence tomography (OCT) analysis. Electroretinogram (ERG) recording was performed to evaluate the retinal function.

### Results

Combination treatment with an autophagy inhibitor, HCQ, and a Fas inhibitor, OCL1204, preserved photoreceptor survival and function in the mouse model of retinitis pigmentosa, P23H.

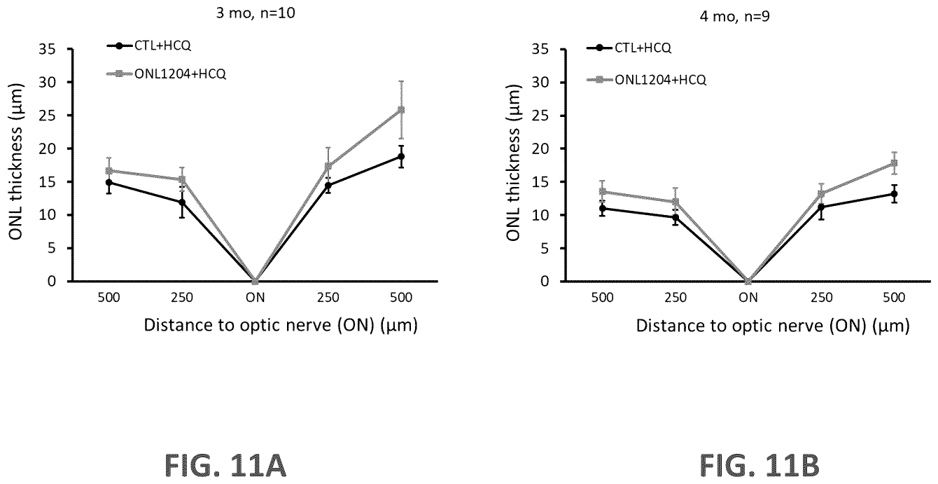

Optic nerve layer (ONL) thickness is a proxy measurement of cell death, with thicker ONLs correlating to less cell death. ONL thickness was measured in the retina in mice that were 3-months old (FIG. 11A) and in mice that were 4-months old (FIG. 11B). P23H mice that were administered combination HCQ and ONL1204 had significantly greater ONL thickness than did P23H mice that were administered HCQ alone. These data indicate that the combination of an autophagy inhibitor and a Fas inhibitor provides greater photoreceptor protection than does the administration of an autophagy inhibitor alone.

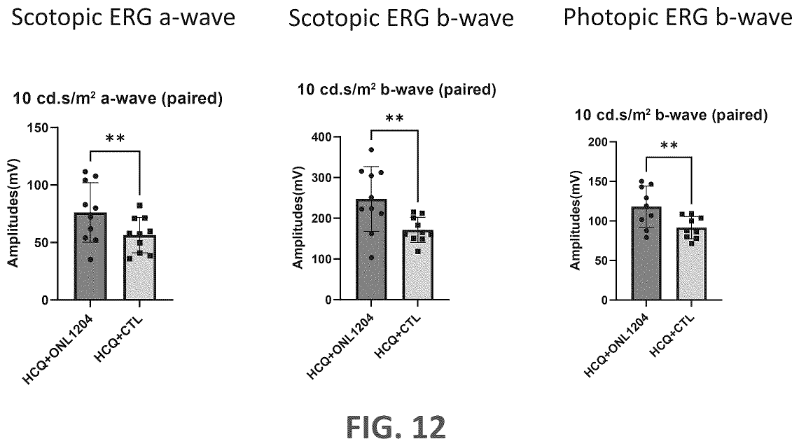

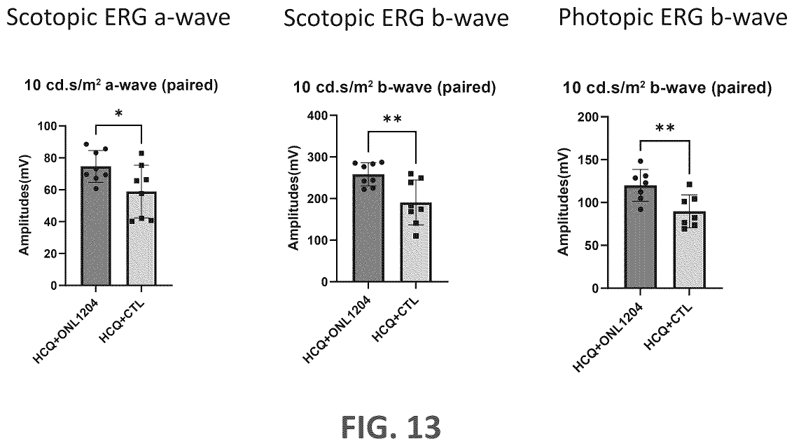

Photoreceptor function of P23H mice was assessed using electroretinograms (ERGs). In both 3-month (FIGS. 12) and 4-month (FIG. 13) old P23H mice scotopic ERG a- waves and b-waves and photopic b-waves had significantly greater amplitude in mice that received combination HCQ and ONL1204 than in mice that received HCQ alone, indicating that autophagy inhibition in combination with Fas inhibition was more effective in preserving photoreceptor function than was autophagy inhibition alone.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

SEQUENCES

| SEQ ID NO | SEQUENCE |
|---|---|
| 1 | HHIYLGAVNYIY |
| 2 | YIYNVAGLYIHH |

1. A method of treating inherited retinal degeneration in an eye of an individual, wherein the method comprises:
administering:
(i) an autophagy inhibitor to the individual; and
(ii) a Fas-inhibiting peptide to the eye, wherein the peptide comprises an amino acid sequence HHIYLGAVNYIY or variant sequence thereof, or a pharmaceutically acceptable salt thereof.

2-3. (canceled)

4. A method of treating retinal cell death in an eye of an individual, wherein the method comprises:
administering:
(i) hydroxychloroquine to the individual; and
(ii) a Fas-inhibiting peptide to the eye, wherein the peptide comprises an amino acid sequence HHIYLGAVNYIY or variant sequence thereof, or a pharmaceutically acceptable salt thereof.

5. The method of claim 4, wherein the retinal cell death comprises photoreceptor cell death, retinal epithelium cell death, or both photoreceptor cell death and retinal epithelium cell death.

6. The method of claim 4, wherein treating retinal cell death comprises neuroprotection of photoreceptors.

7. The method of claim 6, wherein neuroprotection of photoreceptors comprises reducing a decline in photoreceptor loss, reducing a decline in outer nuclear layer thickness, or reducing a decline in retinal nerve fiber layer thickness.

8. The method of claim 4, wherein treating retinal cell death comprises reducing an amount of inflammatory cytokines in the eye.

9. The method of claim 4, wherein treating retinal cell death comprises reducing a decline in outer nuclear layer thickness.

10. The method of claim 4, wherein treating retinal cell death comprises reducing a decline in retinal nerve fiber layer thickness.

11. The method of claim 4, wherein treating inherited retinal degeneration or retinal cell death comprises reducing a decline in visual function in the eye.

12. The method of claim 4, wherein treating inherited retinal degeneration or retinal cell death comprises decreasing a decline in retinal nerve fiber layer thickness.

13. The method of claim 4, wherein the method comprises administering the peptide prior to hydroxychloroquine.

14. The method of claim 4, wherein the method comprises administering hydroxychloroquine prior to the peptide.

15-16. (canceled)

17. The method of claim 4, wherein the hydroxychloroquine is administered orally.

18. The method of claim 4, wherein the peptide or the pharmaceutically acceptable salt thereof has a half-life in the vitreous humor of more than about 30 days.

19-27. (canceled)

28. The method of claim 4, wherein the variant sequence comprises an amino acid substitution or a modification.

29-32. (canceled)

33. The method of claim 4, wherein the peptide has the structure of Formula I: 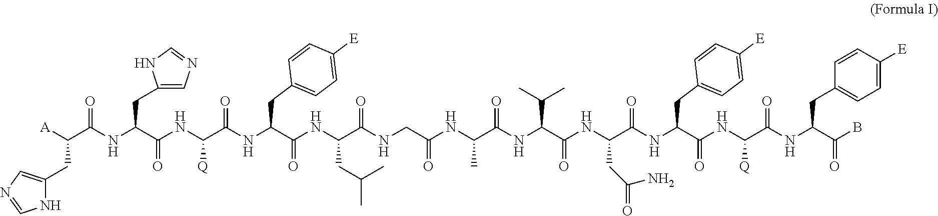
or a pharmaceutically acceptable salt thereof.

34. The method of claim 33, wherein the peptide has the structure of Formula III: 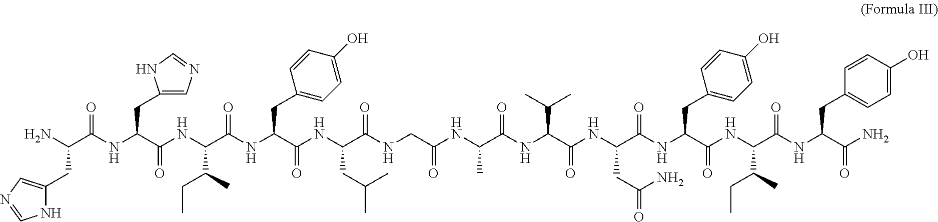 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]
or a pharmaceutically acceptable salt thereof.

35. (canceled)

36. The method of claim 4, wherein the pharmaceutically acceptable salt is an acetate salt or a hydrochloride salt.

37-39. (canceled)

40. The method of claim 4, wherein the method comprises administering a composition comprising the peptide.

41. The method of claim 40, wherein the composition further comprises one or more excipients.

42-49. (canceled)
